(19) United States
(12) Patent Application Publication
(10) Pub. No.: US 2023/0218794 A1
(43) Pub. Date: Jul. 13, 2023

(54) PROCESS AND SYSTEM TO LESSEN HUMAN CORONAVIRUS TRANSMISSION AND SPREAD

(71) Applicant: The Penn State Research Foundation, University, PA (US)

(72) Inventors: Craig Matthew Meyers, Hershey, PA (US); Rena Kass, Hershey, PA (US); David Goldenberg, Hershey, PA (US)

(21) Appl. No.: 17/928,985

(22) PCT Filed: May 14, 2021
(86) PCT No.: PCT/US2021/032357

§ 371 Date: Dec. 1, 2022

Related U.S. Application Data
(60) Provisional application No. 63/039,137, filed on Jun. 15, 2020.
(60) Provisional application No. 63/037,283, filed on Jun. 10, 2020.

(51) Int. Cl. A61L 2/28; A61L 2/18

(52) U.S. Cl. CPC A61L 2/28; A61L 2/18; A61L 2202/25

(57) ABSTRACT

Processes and systems for lessening human spread of coronaviruses can include use of a disinfectants and/or rinses that utilize contact times of up to 2 minutes or less than 2 minutes and also more than 20 seconds. Some embodiments can include gargling a rinse having at least one disinfectant as an active ingredient for up to 2 minutes or for a time period of less than 2 minutes. Other embodiments can include performing a naso-rinse for up to 2 minutes or a time period of less than 2 minutes and also more than 20 seconds with rinse fluid having at least one disinfectant.

### CROSS-REFERENCE TO RELATED APPLICATIONS

The present application claims priority to U.S. Provisional Patent Application Nos. 63/037,283 (filed Jun. 10, 2020) and 63/039,137 (filed Jun. 15, 2020). The entirety of these provisional patent applications are incorporated by reference herein.

### FIELD

The present innovation relates to systems, processes, and methods that can inactivate and/or hinder transmission of coronaviruses.

### BACKGROUND

The rapid spread of COVID 19 cases worldwide in conjunction with its estimated mortality rate make treatment and prevention of this illness imperative. Although vaccine developments and rollout are currently underway, the long-term efficacy of vaccinations and the duration of time it may take to vaccinate a sufficient portion of the world's population is not yet known.

### SUMMARY

The spread of COVID 19 is believed to be primarily through respiratory droplets that can either directly enter an individual's naso- or oropharynx (e.g. nose or mouth), or secondarily be transferred off surfaces by hands touching a fomite and then touching a facial orifice (e.g. eyes, mouth, or nose). Once in the naso- or oropharynx of an individual, the virus is believed to infect the epithelial lining and begins replicating and spreading to other parts of the body. We have developed processes, methods and systems to inactivate and/or hinder the transmission of COVID-19 as well as other coronaviruses that takes into account our understanding of how the virus is believed to infect humans. It is contemplated that the processes, methods and systems we have developed can help mitigate spread an infection in humans and can also help address the ongoing worldwide COVID-19 pandemic.

A process for reducing human coronavirus transmission and/or spread is provide. Embodiments of the process can include testing at least one proposed disinfectant to determine a pre-selected contact time period or pre-selected rinsing time period for each proposed disinfectant to decrease an amount of infectious human coronavirus by at least 90%, 99%, 99.9% or 99.99%, and evaluating results of the testing to determine whether the at least one proposed disinfectant is suitable for a pre-selected environment. The evaluating can include an evaluation of the pre-selected contact time period or the pre-selected rinsing time period determined from the testing of the at least one proposed disinfectant. The process can also include selecting the proposed disinfectant based on the evaluating of the results of the testing for use in a cleaning operation and/or a rinsing operation.

Embodiments of the process can also include performing the rinsing operation and/or performing the cleaning operation. The rinsing operation can include retaining a solution that includes the disinfectant in a mouth of a user for the pre-selected rinsing time period. The rising can also (or alternatively) include rising one or both eyes or one or both nostrils of the use for the pre-selecting rinsing time period. The rinsing can also include rinsing of the nasal passageways of the user for the pre-selected rinsing time period. After the pre-selected rinsing time period, the solution that includes the disinfectant can be spit out of the mouth of the person, blown out the nose, and/or output from the eye or eyes of the user. The performing of the cleaning operation can include contacting a solution that includes the disinfectant on a surface within a building for the pre-selected contact time period. The cleaning operation can be performed within a room of the building. The surface within the building can be a floor, a ceiling, a tabletop, a desktop, a counter, a work surface, or another type of surface.

The testing that is performed can include adding bovine serum albumin (BSA) to a virus suspension that also include a rinse solution comprising a first disinfectant of the at least one proposed disinfectant to form a mixture. The testing can also include incubating the mixture for at least one contact time period. The contact time period can be at least 20 seconds and not more than 5 minutes in some embodiments. The testing can also include assaying viral samples that were formed from the mixture for infectivity. The assaying can utilize a TCID₅₀ assay procedure in some embodiments. Viral samples can be formed by adding a portion of the mixture to a centrifugal filter, adding a neutralizer to the filter, and subsequently centrifuging the viral samples at a pre-selected speed of rotation for a pre-selected centrifuging time period. In some embodiments, the centrifuging is performed at 4,000 revolutions per minute (rpm) for 10 minutes.

In some embodiments of the process, the testing of the at least one proposed disinfectant is performed to determine the pre-selected contact time period or the pre-selected rinsing time period for each proposed disinfectant to decrease the amount of infectious human coronavirus by at least 99%, 99.9% or at least 99.99% (e.g. an amount of infectious human coronavirus remaining after the pre-selected contact time period or pre-selected rinsing time period is reduce by at least 99%, 99.9% or 99.99%).

It should be appreciated that the at least one proposed disinfectant can include a single disinfectant or a plurality of disinfectants (e.g. a first disinfectant, a second disinfectant, a third disinfectant, etc.). The disinfectants can be selected from a group consisting of an ethanol solution, a 70% ethanol solution, a 95% ethanol solution, a glutaraldehyde (GTA) solution, a 2.4% GTA solution, a 3.4% GTA solution, an ortho-phthalaldehyde (OPA) solution, a 0.55% OPA solution, a phenol solution, a paracetic acid-silver (PAA-silver) solution, a 0.25% PAA-silver solution, a 1.2% PAA-silver solution, a hypochlorite solution, and a 0.525% hypochlorite solution, diluted shampoo, diluted baby shampoo, a saline rinse, bromelain, a solution or substance that includes a protein-digesting enzyme mixture derived from the stem, fruit, and juice of the pineapple plant, a solution that includes verbena, common sorrel, elder flower, primula flower and gentain root, an antiseptic oral rinse, and an oral rinse.

In some embodiments, the at least one proposed disinfectant can include at least one of: an ethanol solution, a 70% ethanol solution, a 95% ethanol solution, a glutaraldehyde (GTA) solution, a 2.4% GTA solution, a 3.4% GTA solution, an ortho-phthalaldehyde (OPA) solution, a 0.55% OPA solution, a phenol solution, a paracetic acid-silver (PAA-silver) solution, a 0.25% PAA-silver solution, a 1.2% PAA-silver solution, a hypochlorite solution, a saline solution, a solution or substance that includes a protein-digesting enzyme mixture derived from the stem, fruit, and juice of the pineapple plant, and a 0.525% hypochlorite solution. In such embodiments, the process can also include performing the cleaning operation. The cleaning operation can include contacting a solution that includes the disinfectant on a surface within a building for the pre-selected contact time period. The surface can be a floor, counter, tabletop, desktop, or work surface. Embodiments of such a process can also (or alternatively) include performing the rinsing operation. The rinsing operation can include retaining a solution that includes the disinfectant in a mouth of a user for the pre-selected rinsing time period. The rinsing operation can also include (or alternatively include) rinsing one or both nostrils, one or more nasal passages, and/or one or both eyes of the user for the pre-selected rinsing time period. The solution can then be spit out, blown out, or otherwise output from the user after the pre-selected rinsing time period.

Some embodiments of the process can be designed so that the selecting of the proposed disinfectant based on the evaluating of the results of the testing for use in the cleaning operation and/or the rinsing operation includes selecting a first disinfectant for the cleaning operation and selecting a second disinfectant for the rinsing operation. In such embodiments, the process can include performing the cleaning operation and also performing the rinsing operation. The cleaning operation can include contacting a solution that includes the first disinfectant on a surface within a building for the pre-selected contact time period. The rinsing operation can include retaining a solution that includes the second disinfectant in a mouth of a user for the pre-selected rinsing time period. The solution can then be spit out after the pre-selected rinsing time period. The surface within the building for the cleaning operation can be a floor, a counter, a tabletop, a desktop, or a work surface.

It should be appreciated that the percentages (%) discussed herein can be weight percentages, molar percentages, mass percentages and/or volume percentages. For instance a solution that is 70% ethanol can be 70% by volume ethanol, 70% by mass ethanol, 70 mol. % ethanol, and/or 70% by weight ethanol. The percentage term (%) is used herein to include all of these different options for describing the composition of a particular solution having at least one disinfectant.

Other details, objects, and advantages of our processes and systems for lessening human spread of coronaviruses and methods of making and using the same will become apparent as the following description of certain exemplary embodiments thereof proceeds.

### BRIEF DESCRIPTION OF THE DRAWINGS

Exemplary embodiments of processes and systems for lessening human spread of coronaviruses and methods of making and using the same are shown in the drawings included herewith. It should be understood that like reference characters used in the drawings may identify like components.

FIG. 1 is a table illustrating determined efficacies for different rinses and/or fluids that can be used in embodiments of our process and system.

FIG. 2 is a flow chart first exemplary process for lessening human spread of coronaviruses.

### DETAILED DESCRIPTION OF EXEMPLARY EMBODIMENTS

We determined that we can test the efficacy of common disinfectants for their ability to inactivate Coronaviruses. Using a BSL2 level Coronavirus as a surrogate model, we tested the ability of common hospital disinfectant to inactivate Coronavirus. Table 1 below lists common disinfectants that we utilized in this testing work:

TABLE 1
Disinfectants
70% Ethanol solution
95% Ethanol solution
2.4% Glutaraldehyde (GTA) solution
3.4% GTA solution
0.55% ortho-phthalaldehyde (OPA) solution
Phenol solution
0.25% paracetic acid-silver (PAA-silver) solution
1.2% PAA-silver solution
0.525% hypochlorite solution
UV-C light (e.g. via a UVC lamp)

We determined that a suspension assay can be used to measure disinfectant efficacy using contact times from 1 minute to 45 minutes. A carrier method assay can also be used to measure disinfectant efficacy using contact times from 1 minute to 45 minutes. Carriers can be made out of the materials use to make cell phones (i.e. the glass front and the plastic cover) as a surrogate for fomite spread. Negative and positive controls can be used as well.

We determined that can identify common disinfectants that can inactivate coronavirus with their required contact times. We can also demonstrate the practicality of inactivating the virus on fomites that are capable of transmitting the virus from one host to another based on the testing methodology we developed.

We also evaluated intransal and oropharyngeal rinses. Table 2, below, identifies different rinses we evaluated. We also evaluated the rinses of Table 2 because we determined that we could test the efficacy of intranasal and oropharyngeal rinses for their ability to inactivate Coronavirus. Using a BSL2 level Coronavirus as a surrogate model, we can test the ability of commonly used intranasal and oropharyngeal rinses (TABLE 2). We can use a suspension assay to measure efficacy using contact times from 30 seconds to 5 minutes. Rinses that showed efficacy in the suspension assay can also be tested for efficacy in 2-D and 3-D primary epithelial tissue culture models. Coronavirus can be applied to 2-D epithelial culture and/or 3-D primary epithelial culture model with increasing multiplicities of infection (MOI). Rinses can then be applied with contact times of 30 seconds to 5 minutes. The cells can then be immediately harvested and tested for infectivity.

TABLE 2
Intranasal and Oropharyngeal Rinses
Diluted Baby Shampoo
Saline rinses of different saline contents (e.g. solutions that
include salt and water).
Sinupret (and similar compounds, which include solutions
that include verbena, common sorrel, elder flower, primula
flower and gentain root)
Bromelain or other similar substance (e.g. a solution or
substance that can include a protein-digesting enzyme
mixture derived from the stem, fruit, and juice of the
pineapple plant)
Oral rinses (different varieties of oral antiseptic rinses)

We determined that the testing we had developed can identify intranasal and oropharyngeal washes capable of inactivating the virus. This can be demonstrated both in a suspension model and in physiologically relevant tissue models as noted herein.

A suspension assay can be used to measure disinfectant efficacy using contact times from 1 minute to 45 minutes. A carrier method assay can be used to measure disinfectant efficacy using contact times from 1 minute to 45 minutes. This testing can identify intranasal and oropharyngeal washes capable of inactivating coronaviruses (e.g. COVID-19 as well as other coronaviruses). This can be demonstrated both in a suspension model and in physiologically relevant tissue models as discussed herein.

We determined that the testing and methodology discussed herein can define which disinfectants can inactivate the COVID-19 virus as well as other coronaviruses both in suspension and on fomites and necessary contact times that may be required. We can demonstrate the potential of intranasal and oral rinses to prevent transmission as well.

After suitable disinfectants or rinses with efficacy are determined via testing, identified reagents using actual COVID 19 can be used to further test the efficacy in the ferret in vivo model or via other types of models. In additional testing, human subjects could be utilized. For instance, the collection of tissue specimens that can be used to create primary epithelial cell lines can be obtained from humans. These specimens can be removed from human subjects. The tissue collected from standard surgeries is tissue that that can be de-identified and would normally be discarded and this tissue could be utilized for such testing.

With coronavirus diseases and the COVID-19 pandemic, we have determined that it is important to identify procedures that can lower the potential transmission and spread of human coronavirus (HCoV). Embodiments of our process and systems disclosed herein has been determined as being able to effectively demonstrating that common over-the-counter naso-rinse and oro-rinse products can be utilized with a high efficacy in killing HCoV.

As discussed herein, we tested the efficacy of multiple over-the-counter naso-rinse and oro-rinse products for their ability to kill HCoV using contact times of 2 min, 1 min, and 30 sec. Appropriate neutralizers were added to inactivate the solutions and the formed samples were filtered centrifuged at a pre-selected speed of rotation for a pre-selected centrifuging time period. Reductions in titers were measured by using the tissue culture and infectious dose 50 (TCID₅₀) assay.

As discussed herein, some of the over-the-counter naso-rinse and oro-rinse products show high efficacy in killing HCoV. These include 5% Povidone-Iodine, Crest Pro Health, Listerine, Listerine Ultra, CVS Antiseptic Mouth Wash, Equate Antiseptic, and a 1% dilution of Johnson & Johnson Baby Shampoo. While other showed lower levels of killing including, Peroxide Sore Mouth, H202 Antiseptic, Orajel Antiseptic Rinse, and Neti Pot.

We have determined that several over-the-counter naso-rinse and oro-rinse products can extensively lower the number of infectious HCoV greatly lowering the potential for transmission and spread. This is important for the public in general but also has specific importance in areas such as medical examiners, surgeons, and dentists where gargling and nebulizing one of these products would greatly lower the risks.

Materials and Methods Used in Conducted Testing

Cell Lines, Cell Culture and Virus.

Huh7 cells were grown in Dulbecco Modified Eagle's Medium (DMEM) supplemented with 10% fetal bovine serum (FBS) (DMEM10) and 100 U/ml pen/strep, and the cells grown in 5% CO₂ at 37° C. Infectious stocks of Human Coronavirus 229e (HCoV 229e) were prepared by seeding T75 flasks with 7×10⁶ Huh7 cells and incubated overnight. On the following day the media was changed to DMEM with 2% FBS (DMEM2) and a multiplicity of infection (MOI) of 0.01 of virus was added to the flasks. The infected flasks were incubated for two days in 5% CO₂ at 35° C. On the second day the flasks were frozen at −80° C. for at least 1 h, then thawed in a 37° C. water bath taking care to remove them from the water bath before they were completely thawed. Thawing was then completed at room temperature. The cell suspensions were transferred to a 15 ml polypropylene tube and sonicated on ice in a cup sonicator at 100 watts peak envelope power, 3 bursts of 20 seconds each. The lysates were clarified by centrifugation at 3,000 rpm for 10 minutes at 4° C. and the supernatant poured into a fresh 15 ml tube. Virus solutions were aliquoted into 8-0.5 ml portions and several smaller aliquots then frozen for long term storage at −80° C. One of the smaller aliquots was used to determine the titer of the stock by the tissue culture infectious dose 50 (TCID₅₀) assay.

TCID₅₀ Assay

Huh7 cells were harvested, counted, and re-suspended into DMEM2 to a concentration of 1.5×10⁶ cells/ml. Then 100 μl of the cell suspension is added to each well of the 96-well plate. Plates were incubated overnight in 5% CO₂ at 37° C. Serial 10-fold dilutions of virus were added to each column of wells containing cells. An extra row of mock-infected cells were included across the bottom. The plates were then incubated for 3 days in 5% CO₂ at 35° C. On the third day the wells were examined for the presence of cytopathic effects (CPE) and the TCID₅₀ calculation was done using the Reed-Meunch method based off the number of wells positive for CPE at each dilution.

Naso-Rinse and Oro-Rinse Products

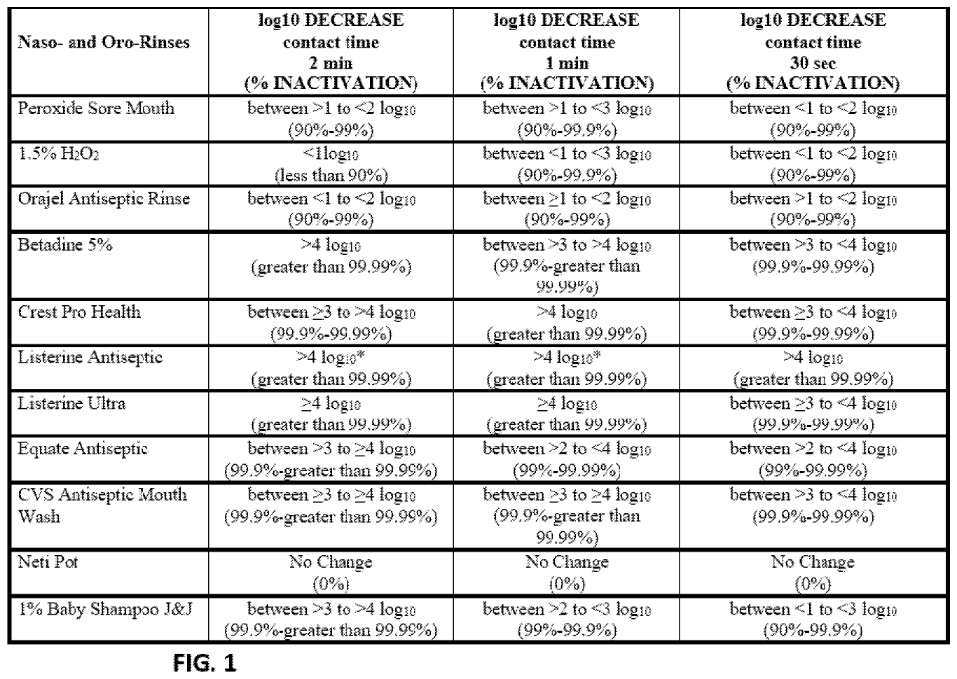

Over the counter naso-rinse and oro-rinse products used in the study are shown in the table of FIG. 1. Two hundred μl of an organic load or soil of 5% bovine serum albumin (BSA) was added to the virus suspension 1 ml of the rinse solution. The rinse/virus mixes were then incubated at room temperature for contact times of 2 minutes, 1 minute, and 30 seconds. The solutions were added to a 15 ml Amicon Ultra centrifugal filter (100,000 MW cut-off [MWCO]) and immediately 2 ml of neutralizer was added to the filters. The filters that contained the viral samples were then centrifuged at a pre-selected speed of rotation for a pre-selected centrifuging time period. For our experiments reported in FIG. 1, the pre-selected speed of rotation was 4,000 revolutions per minute (rpm) and the pre-selected centrifuging time period was 10 minutes. The viral samples were then assayed for infectivity using the TCID₅₀ method discussed herein. Three or more replicate assays were done for each rinse and contact time. Controls of untreated virus were included for every set of assays performed.

FIG. 1 shows the efficacy of each of the naso-rinse and oro-rinse products utilized in this conducted testing. The asterisk included in different table items in FIG. 1 indicate that there was no detection of any remaining infectious virus. The Log₁₀ reductions noted in FIG. 1 represent the following percent decreases in infectious virus as shown in Table 3 (e.g. a reduction of at least 90% of infectious virus from the original amount was detected for a Log₁₀ decrease of 1, a reduction of at least 99% of infectious virus from the original amount was detected for a Log₁₀ decrease of 2, a reduction of at least 99.9% of infectious virus from the original amount was detected for a Log₁₀ decrease of 3, and a reduction of at least 99.99% of infectious virus from the original amount was detected for a Log₁₀ decrease of 4).

TABLE 3
Decreases in HCoV Infectiousness

| Log₁₀ Decrease | Percent Decrease in infectious HCoV |
|---|---|
| 4 | 99.99% |
| 3 | 99.9% |
| 2 | 99% |
| 1 | 90% |

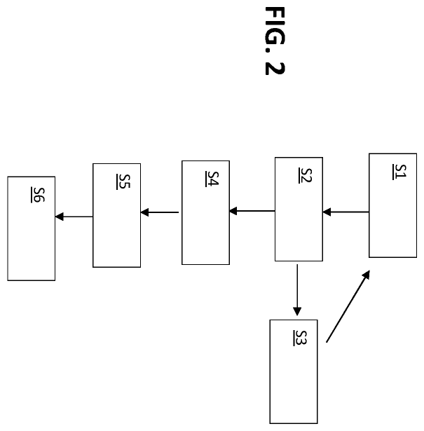

Based on our conducted testing and evaluations as discussed herein, we have developed methods and processes for evaluation of disinfection systems and processes that can be employed to reduce or eliminated human coronavirus transmission as well as methods and processes for reducing or eliminating human coronavirus transmission. FIG. 2 illustrates an exemplary flow chart of such a system.

In a first step S1, a proposed disinfectant can be tested to determine a pre-selected contact time period of a proposed disinfectant solution and/or a pre-selected rinsing time period of the proposed disinfectant solution. Such testing can include, for example, the testing discussed herein that was performed in conjunction with obtaining the results shown in FIG. 1. The disinfectant solution can be a liquid or fluid having at least one disinfectant active ingredient (e.g. an alcohol, an antiseptic agent, etc.).

In a second step S2, the testing results for the proposed disinfectant(s) can be evaluated to select one or more disinfectants that were determined to best meet a particular need for a pre-selected environment or activity (e.g. daycare cleaning after children have left daycare, cleaning of an office at a particular time, having personnel perform certain rinses at one or more times a day during a work day, etc.). This selection can be based on the pre-selected contact.

The evaluation performed in the second step S2 can result in a third step S3 being performed to identify other proposed disinfectant(s) to undergo further evaluation by repeating of the first and second steps to account for the new proposed disinfectants. This third step S3 can occur in the event the testing results evaluated in the second step S2 indicate that the proposed disinfectants may not be sufficiently suitable for a particular desired objective or may result in incurring a cost that is too high for the particular task or environment in which the disinfectant would be used.

In a fourth step S4 that can be performed in response to the test results evaluated in the second step S2 can indicate at least one tested disinfectant option would be suitable for a proposed task or environment, the one or more disinfectants found to be acceptable can be sourced for being acquired and used. Cleaning protocols or other type of rinse or use protocols can be developed to guide use of the one or more disinfectants to perform at least one disinfection operation (e.g. a rinse to be performed for a pre-selected rinsing time period by different personnel at different times of day during a working day and/or utilization of a disinfectant to be performed when cleaning a particular object or work space for a pre-selected duration of contact time with the object/space to be cleaned, etc.). A pre-selected rinsing time period or pre-selected duration of contact time period can be determined in the fourth step S4 based on the testing results evaluated in the second step S2.

In a fifth step S5, the cleaning and/or rinse protocols can be implemented by personnel to perform a cleaning or rinsing task in accordance with the protocol(s) developed in the fourth step S4. For example, the implementation of the cleaning and/or rinsing protocol(s) can include performing a rinsing operation and/or a cleaning operation. The rinsing operation can include retaining a solution that includes a selected disinfectant in a mouth of a user for the pre-selected rinsing time period. A person can then spit the solution that includes the disinfectant out of the person's mouth after the pre-selected rinsing time period. The person can swish or otherwise manipulate the solution within the mouth during the pre-selected rinsing time period as well. The rinsing operation can also (or alternatively) including rinsing of one or both nostrils of the user, one or both eyes of the user and/or one or both nasal passages of the user. The rinsing can occur for the pre-selected rinsing time period. After that time period has elapsed, the solution can be output from the user (e.g. blown out of nostrils, otherwise output, etc.).

Performing the cleaning operation can include contacting a solution that includes the disinfectant on a surface within a building for the pre-selected contact time period. The surface within the building can include, for example, a floor, a counter, a tabletop, a desktop, or a work surface within a room of a building.

In some embodiments, there may be a rinsing operation and a cleaning operation that are both under consideration. In such embodiment, a first disinfectant can be selected for the cleaning operation and a second disinfectant can be selected for the rinsing operation. In embodiments in which the rinsing operation can include rinsing of the mouth, eyes, and/or nose, there may be additional disinfectants (e.g. a third disinfectant for eye rinsing and a fourth disinfectant for nasal rinsing, etc.).

This activity (e.g. the cleaning operation and/or rinsing operation) can also be monitored to evaluate its efficacy in a sixth step S6 so that the protocols that are developed can be revised as may be needed to help address unexpected or unforeseen problems or issues that may be identified during the implementation that occurs in the fifth step S5. In some situations, the sixth step S6 may result in determining that the selected disinfectant is no longer acceptable due to one or more unforeseen difficulties or problems. If this occurs, the process may be restarted at the first step S1 to evaluate other potential replacement disinfectants.

It should be appreciated that modifications to the embodiments explicitly shown and discussed herein can be made to meet a particular set of design objectives or a particular set of design criteria. For instance, it is contemplated that a particular feature described, either individually or as part of an embodiment, can be combined with other individually described features, or parts of other embodiments. The elements and acts of the various embodiments described herein can therefore be combined to provide further embodiments. Thus, while certain exemplary embodiments of the processes and systems for lessening human spread of coronaviruses and methods of making and using the same have been shown and described above, it is to be distinctly understood that the invention is not limited thereto but may be otherwise variously embodied and practiced within the scope of the following claims.

What is claimed is:

1. A process for reducing human coronavirus transmission and/or spread, the process comprising:
testing at least one proposed disinfectant to determine a pre-selected contact time period or pre-selected rinsing time period for each proposed disinfectant to decrease an amount of infectious human coronavirus by at least 90%;
evaluating results of the testing to determine whether the at least one proposed disinfectant is suitable for a pre-selected environment, the evaluating including an evaluation of the pre-selected contact time period or the pre-selected rinsing time period determined from the testing of the at least one proposed disinfectant;
selecting the proposed disinfectant based on the evaluating of the results of the testing for use in a cleaning operation and/or a rinsing operation.

2. The process of claim 1, wherein the process also comprises:
performing the rinsing operation, the rinsing operation comprising retaining a solution that includes the disinfectant in a mouth of a user for the pre-selected rinsing time period.

3. The process of claim 1, wherein the process also comprises:
performing the cleaning operation, the cleaning operation comprising contacting a solution that includes the disinfectant on a surface within a building for the pre-selected contact time period.

4. The process of claim 3, wherein the cleaning operation is performed within a room of the building.

5. The process of claim 1, wherein the testing includes:
adding bovine serum albumin (BSA) to a virus suspension that also include a rinse solution comprising a first disinfectant of the at least one proposed disinfectant to form a mixture;
incubating the mixture for at least one contact time period, the contact time period being at least 20 seconds and not more than 5 minutes; and
assaying viral samples that were formed from the mixture for infectivity.

6. The process of claim 5, wherein the assaying utilizes a TCID₅₀ assay procedure.

7. The process of claim 5, wherein the viral samples are formed by adding a portion of the mixture to a centrifugal filter, adding a neutralizer to the filter, and subsequently centrifuging the viral samples at a pre-selected speed of rotation for a pre-selected centrifuging time period.

8. The process of claim 7, wherein the centrifuging is performed at 4,000 revolutions per minute (rpm) for 10 minutes.

9. The process of claim 1, wherein the testing of the at least one proposed disinfectant is performed to determine the pre-selected contact time period or the pre-selected rinsing time period for each proposed disinfectant to decrease the amount of infectious human coronavirus by at least 99%.

10. The process of claim 1, wherein the testing of the at least one proposed disinfectant is performed to determine the pre-selected contact time period or the pre-selected rinsing time period for each proposed disinfectant to decrease the amount of infectious human coronavirus by at least 99.9%.

11. The process of claim 1, wherein the testing of the at least one proposed disinfectant is performed to determine the pre-selected contact time period or the pre-selected rinsing time period for each proposed disinfectant to decrease the amount of infectious human coronavirus by at least 99.99%.

12. The process of claim 1, wherein the at least one proposed disinfectant include a plurality of disinfectants.

13. The process of claim 12, wherein the disinfectants are selected from the group consisting of an ethanol solution, a 70% ethanol solution, a 95% ethanol solution, a glutaraldehyde (GTA) solution, a 2.4% GTA solution, a 3.4% GTA solution, an ortho-phthalaldehyde (OPA) solution, a 0.55% OPA solution, a phenol solution, a paracetic acid-silver (PAA-silver) solution, a 0.25% PAA-silver solution, a 1.2% PAA-silver solution, a hypochlorite solution, and a 0.525% hypochlorite solution, diluted shampoo, diluted baby shampoo, a saline rinse, bromelain, a solution or substance that includes a protein-digesting enzyme mixture derived from the stem, fruit, and juice of the pineapple plant, a solution that includes verbena, common sorrel, elder flower, primula flower and gentain root, an antiseptic oral rinse, and an oral rinse.

14. The process of claim 1 wherein the at least one proposed disinfectant includes at least one of: an ethanol solution, a 70% ethanol solution, a 95% ethanol solution, a glutaraldehyde (GTA) solution, a 2.4% GTA solution, a 3.4% GTA solution, an ortho-phthalaldehyde (OPA) solution, a 0.55% OPA solution, a phenol solution, a paracetic acid-silver (PAA-silver) solution, a 0.25% PAA-silver solution, a 1.2% PAA-silver solution, a hypochlorite solution, a saline solution, a solution or substance that includes a protein-digesting enzyme mixture derived from the stem, fruit, and juice of the pineapple plant, and a 0.525% hypochlorite solution.

15. The process of claim 14, wherein the process also comprises:
performing the cleaning operation, the cleaning operation comprising contacting a solution that includes the disinfectant on a surface within a building for the pre-selected contact time period.

16. The process of claim 15, wherein the surface is a floor, a counter, a tabletop, a desktop, or a work surface.

17. The process of claim 14, wherein the process also comprises:
performing the rinsing operation, the rinsing operation comprising retaining a solution that includes the disinfectant in a mouth of a user for the pre-selected rinsing time period.

18. The process of claim 14, wherein the selecting the proposed disinfectant based on the evaluating of the results of the testing for use in the cleaning operation and/or the rinsing operation includes selecting a first disinfectant for the cleaning operation and selecting a second disinfectant for the rinsing operation, the process also comprising:
performing the cleaning operation, the cleaning operation comprising contacting a solution that includes the first disinfectant on a surface within a building for the pre-selected contact time period; and
performing the rinsing operation, the rinsing operation comprising retaining a solution that includes the second disinfectant in a mouth of a user for the pre-selected rinsing time period.

19. The process of claim 18, wherein the surface is a floor, a counter, a tabletop, a desktop, or a work surface.

20. The process of claim 19, wherein the performing of the rinsing operation also includes spitting out the solution that includes the second disinfectant after the pre-selected rinsing time period has passed.
